(19) United States
(12) Patent Application Publication
(10) Pub. No.: US 2016/0073701 A1
(43) Pub. Date: Mar. 17, 2016

(54) Wearable Electrical Connectors

(71) Applicant: Penn Engineering & Manufacturing Corp., Danboro, PA (US)

(72) Inventor: Michael J. Maloney, Doylestown, PA (US)

(21) Appl. No.: 14/856,529

(22) Filed: Sep. 16, 2015

Related U.S. Application Data
(60) Provisional application No. 62/051,360, filed on Sep. 17, 2014.

(51) Int. Cl. A41D 1/00; H01R 12/70; H01R 13/73; H01R 4/02

(52) U.S. Cl. CPC A41D 1/005; H01R 4/02; H01R 12/7076; H01R 13/73

(57) ABSTRACT

An wearable electronic connection assembly including a circuit board connected to a garment. The connector of the assembly has, from top to bottom, a tack pin having a head, a shank, and clinch attachment means with an undercut for receiving the cold flow of metal. The head of the pin passes through a hole in the circuit board and is in contact with a top surface of the circuit board. The shank of the pin extends farther downward through a layer of fabric beneath the circuit board. A ring is disposed beneath the fabric layer into which the tack pin is clinched forcing material of the ring into the undercut. A wire is attached to the ring which is electrically connected to the circuit board through its connection to the electrically-conductive pin. The assembly may include a plurality of pins, each of which extends through a separate hole in the circuit board and is clinched by a separate clinch ring.

### RELATED APPLICATION

This patent application is a non-provisional of provisional patent application No. 62/051,361 filed Sep. 17, 2014 entitled “Wearable Electrical Connectors”, priority from which is hereby claimed.

### FIELD OF THE INVENTION

The invention relates to electrical connectors and assemblies of wearable connectors. More specifically, it relates to devices that connect, both mechanically and electrically, electronic devices to the fabric of garments.

### BACKGROUND OF THE INVENTION

Wearable electronics are electronic devices incorporated into clothing to make the device more accessible to users. Examples of wearable electronics include: interface devices such as glasses and earphones; processing devices such as music players, cell phones, laptops and tablets; input devices such as health monitors, pressure sensors or gait/pace sensors in shoes; and, GPS location sensors. Other types of wearable electronics on the horizon might include blood sugar monitors, heart rate monitors, hydration sensors, and temperature sensors. Still other types of wearable electronic could include comfort control devices that circulate cooling media within the actual clothing, or devices that constrict the cloth itself to aid blood circulation from the extremities to vital organs

Wearable electronics must be mechanically connected/secured to or within the garment itself. Additionally, wearable electronics must be electrically connected to a conduction lead, such as a flexible wire, which may be connected to a power source, another wearable electronic, processing unit, or other electrical device. The conduction leads must also be coupled together and attached within the fabric. Furthermore, the wearable electronics on separate garments, such as a shirt and pants, should also be electrically coupled together easily.

Some current methods for attaching a conduction lead to wearable connectors include rivets, direct soldering to the electronics, and actual stitching into the fabric around conductors, with thread. Rivets were not originally designed for and are unreliable for attaching flexible wire because the wires frequently loosen and fail. Manually soldering each conduction lead during clothing assembly is effective, but very slow for a high volume production process. Manually stitching each conduction lead is also very slow although very effective. Manually stitching a processing board to clothing is effective but time consuming. Furthermore, the threads will eventually fail due to stress, wear and fatigue. There is therefore a need for an efficient, high-volume manufacturing method and device to mechanically and electrically connect wearable electronics to garments.

### SUMMARY OF THE INVENTION

The present invention provides surface-mount connectors for mechanically and electrically connecting wearable electronics to garments. The connectors can be installed using existing machinery designed for soldering electronic components to electronics boards and to also attach mounting hardware, such as standoffs and threaded nuts. The connectors include a tack pin that attaches two panels, such as a circuit panel and a fabric panel, together by clinching into the base material of one component of the connector. The present invention utilizes tack pin technology wherein the tack pin head is surface-mount soldered to the wearable electronic panel or peripheral connections. The tack pin is used in combination with a secondary collar such as a clinch ring on the opposite side of the garment fabric to clinch one end of the conductor in place.

The invention includes at least three different embodiments of connectors, which include; a simple ring to which a conductor lead is soldered; a slotted bulb pressed into a notched clinch ring into which the electrical lead is placed; and, a tack pin with an undercut large enough to wrap a small wire within it. All embodiments of the connector provide a high-speed, high-volume electronic assembly and garment attachment method.

In one preferred embodiment of the invention, the connector of the electronic connection assembly comprises from top to bottom, a tack pin having a head, a shank, and clinch attachment means with an undercut for receiving the cold flow of metal. The head of the pin passes through a hole in the circuit board and sits in contact with a top surface of the circuit board. The shank of the pin extends farther downward through a layer of fabric beneath the circuit board. A ring is disposed beneath the fabric layer into which the tack pin is pressed, thereby forcing material of the ring into the undercut of the tack pin. A conduction lead (wire) is mechanically and electrically connected to the ring, which is also mechanically and electrically connected to the circuit board through the tack pin. A plurality of connectors may be used to fasten the wearable electronic to the garment. Each connector extends through a separate hole in the electronic, such as the circuit board, and is clinched to a separate ring. In one preferred embodiment, the tack pin has a slot at the bottom and the ring has a peripheral groove through which the conductor lead passes. Alternatively, the wire may occupy the undercut of the pin. Connections between the pin, the wire and the circuit board may all be soldered. A single circuit board can hold a plurality of pins, which attach though the fabric and into the rings.

Other advantages will follow from the foregoing explanation and the following drawings and description of the invention. The preferred embodiment of the invention will provide one of skill in the art with a full understanding of what has been invented. It will thereby be appreciated that the object of the invention to devise an electronic circuit affixed to a garment with various advantages over the prior art has been achieved.

### BRIEF DESCRIPTION OF THE DRAWINGS

FIGS. 1a and 1b are cross sections of a connector assembly in accordance with a preferred embodiment of the invention shown before and after, respectively, press connection between a wearable electronic and a fabric;

FIG. 2 is a top plan view of a wearable electric device connected to a fabric using a plurality of connector assemblies in accordance with a preferred embodiment of the invention;

FIG. 3 is a front elevation of the device shown in FIG. 2;

FIG. 4 is a bottom plan view of the device shown in FIG. 3;

FIG. 5 is a fragmentary, exploded perspective of the connector assembly show in FIG. 1;

FIG. 6 is a fragmentary side elevation of the connector assembly shown in FIG. 1;

FIG. 7 is a cross section taken along lines 7-7 of FIG. 2;

FIG. 8 is an fragmentary, exploded perspective of a connector assembly in accordance with another embodiment of the invention;

FIG. 9 is a fragmentary perspective of the connector assembly of FIG. 8;

FIG. 10 is a cross section taken along lines 10-10 of FIG. 9;

FIG. 11 is a cross section taken along lines 11-11 of FIG. 9;

FIG. 12 is a fragmentary, exploded perspective of a connector assembly in accordance with yet another embodiment of the invention;

FIG. 13 is a perspective of the connector assembly of FIG. 12 in the assembled condition; and,

FIG. 14 is a cross section taken along lines 14-14 of FIG. 13.

### DETAILED DESCRIPTION OF THE PREFERRED EMBODIMENTS

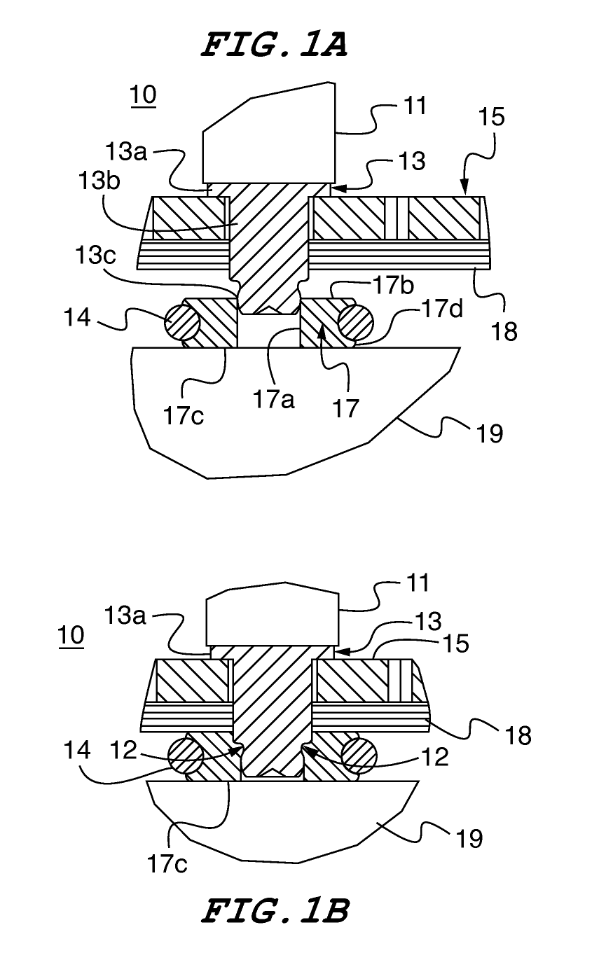

A connector in accordance with a preferred embodiment is shown in FIGS. 1-7 and is designated generally by reference numeral 10. The connector of the assembly mechanically and electrically joins a wearable electronic 15 to a fabric layer 18. The connector generally comprises a tack pin 13, a metal clinch ring 17 and a conductor 14 integrally connected to the ring 17. The tack pin 13 has a head 13a, a shank 13b, a bulbous end 13c, and clinch attachment means intermediate the head 13a and the bulbous end 13c. In a preferred embodiment, the clinch attachment means comprises an undercut 12 in the shank 13b for receiving the cold flow of metal. The head 13a of the tack pin 13 is constructed and arranged to be soldered to the surface of the wearable electronic 15, such as the circuit board, using known surface mount technology. The shank 13b of the tack pin 13 extends through a hole in the wearable electronic 15.

In a preferred embodiment, the metal clinch ring 17 has a central, cylindrical bore 17a, annular, flat top and bottom surfaces 17b, 17c, respectively, a perimeter surface 17d, and a concave race 17e in the perimeter surface 17d. In a preferred embodiment, the concave race 17e is generally hemispherical and has a shape that compliments the outer surface of the conductor lead 14. The clinch ring 17 is made of a metal that can be deformed by the tack pin 15 so that metal from the clinch ring 17 flows into the undercut 12 during installation.

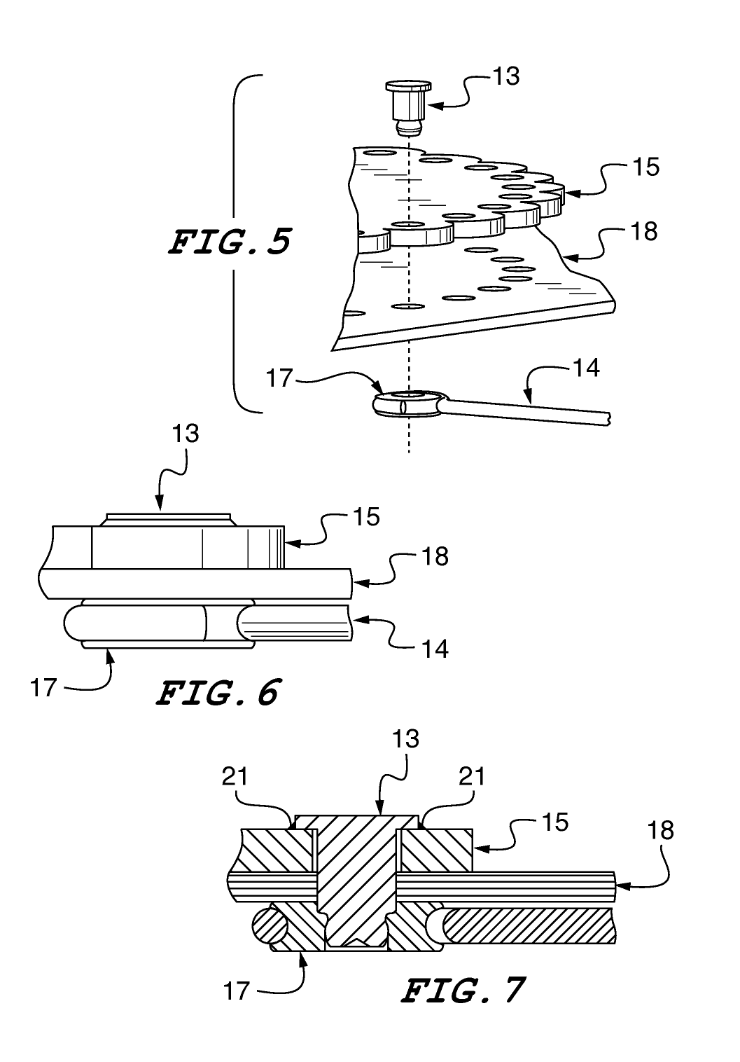

In the preferred embodiment shown in FIGS. 1-7, the conduction lead comprises a strand of metal wire having a circular cross section. As best seen in FIG. 5, one end portion of the wire loops around the clinch ring 17 and sits in the race 17e. Preferably, the wire has an outer profile that closely matches the profile of the race 17e. As seen in FIG. 5, the wire need not extend around entire perimeter of the clinch ring 17. However, in this embodiment, the wire should be tightly wound around the clinch ring 17 to create a good mechanical and electrical connection between the two components. Alternatively, the wire may be soldered in the race. The remainder of the wire extends outwardly from the clinch ring.

Installation of the connector 10 is illustrated in FIGS. 1a and 1b. Referring to FIG. 1a, a tack pin 13 is shown extending through aligned apertures in the circuit board of a wearable electronic 15 and a fabric layer 18. A clinch ring 17 is partially installed on the shank of the tack pin 13. With the clinch ring 17 supported by an anvil 19, a press 11 engages the head 13a of the tack pin and forces the shank 13b into the clinch ring as shown in Fig. lb. During installation, the shoulder of the undercut 12 deforms the clinch ring and forces metal from the circuit board into the undercut 12. This process attaches the circuit board 15 to the fabric 18. Preferably, the conductor 14 is soldered in the race and the tack pin head 13a is soldered to the circuit board. When the clinch ring 17 is tightly clinched to the tack pin 13, a reliable electrical connection is completed between the conductor lead 14 and the circuit board 15. These surface mount installation steps can be achieved using known automated manufacturing equipment. Therefore, the connector 10 will make high-volume connection of wearable electronics to fabrics very economical.

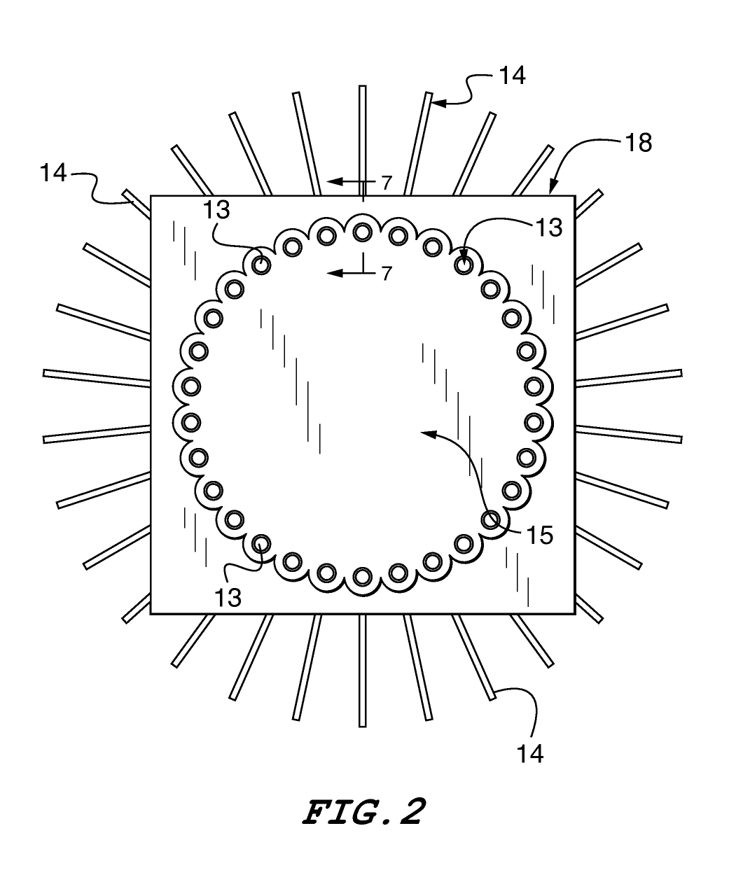

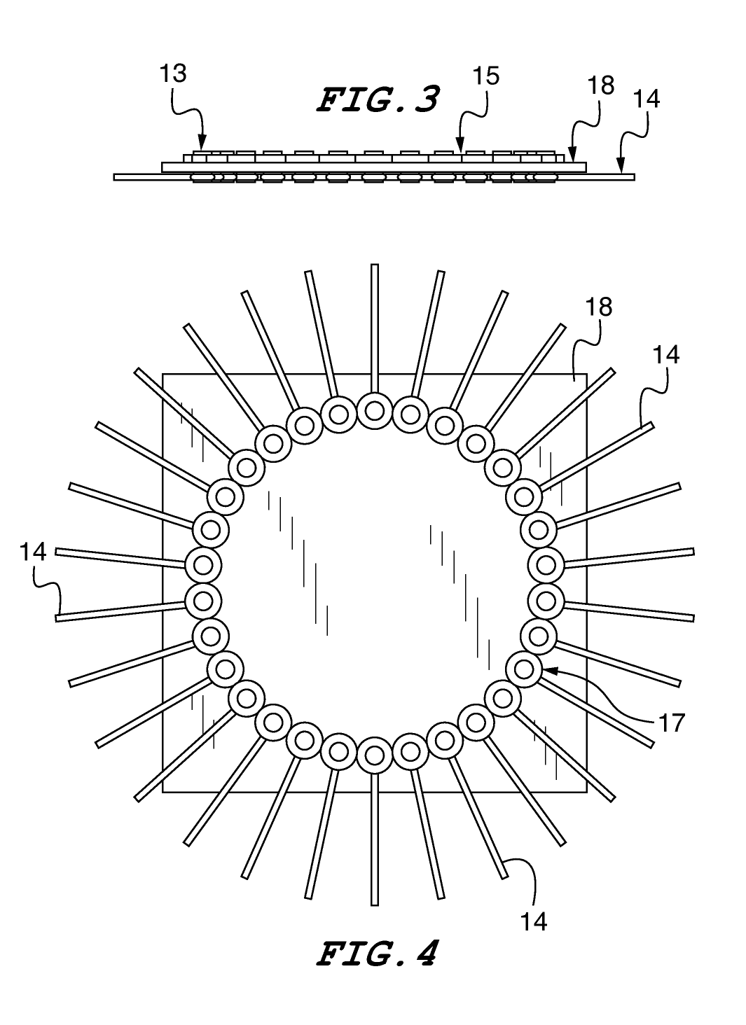

FIGS. 2 through 4 depict one type of circuit board that can be connected using the connectors of the present invention. FIGS. 2-4 show a substantially-circular circuit board 15 connected to a section of fabric 18 with a plurality of tack pins 13. Conductor leads 14 extend radially from each clinch ring 17 as best seen in FIG. 4.

FIGS. 5 through 7 show the connector 10 in greater detail. FIG. 7 shows the solder 21 between the tack pin head 13 and the circuit board 15. A section of fabric 18 is interposed between the circuit board 15 and the clinch ring 17.

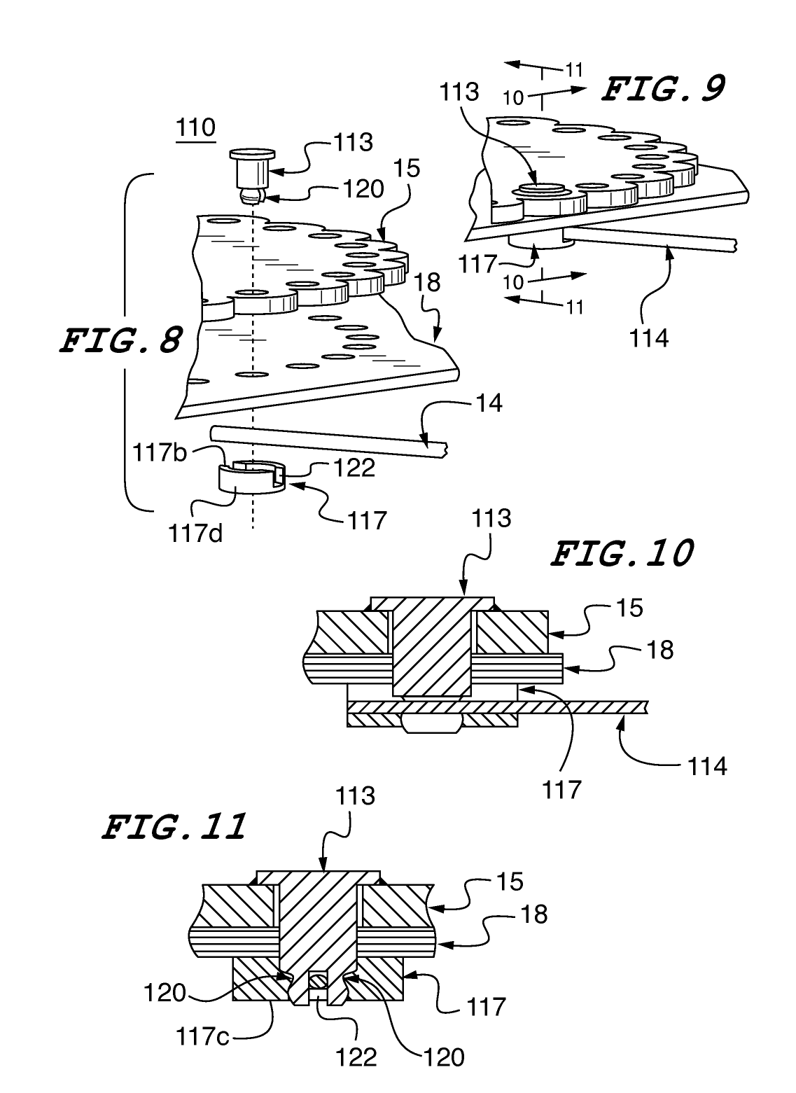

Another preferred embodiment of the invention is shown in FIGS. 8 through 11 and is designated generally by reference numeral 110. The connector of the assembly mechanically and electrically joins a wearable electronic 15 to a fabric layer 18. The connector generally comprises a tack pin 113, a metal clinch ring 117 and a conductor lead 114. The tack pin 113 has a head 113a, a shank 113b, a bulbous end 113c, and clinch attachment means intermediate the head 113a and the bulbous end 113c. The tack pin 113 has a construction similar to the tack pin 13 illustrated and described above with respect to FIGS. 1-7. However, in this embodiment, the tack pin 113 has a slot 120 in the end along the diameter.

In this preferred embodiment, the metal clinch ring 117 has a central, cylindrical bore, annular, flat top and bottom surfaces 117b, 117c, respectively, and perimeter surface 117d. However, in this embodiment, perimeter surface 117d does not have a race. Instead, the clinch ring 117 has a slot 122 in the top surface 117b which extends along the diameter of the clinch ring 117. The slot 122 in the clinch ring is constructed and arranged to align with the slot 120 in the tack pin 113. The slot 122 provides clearance for the conductor lead 114 wire (described below) so that the lead is not sheared off during installation.

The conduction lead 114 comprises a strand of metal wire having a circular cross section. As best seen in FIGS. 9 and 11, the diameter of the wire is slightly smaller than the width of the slots 120, 122.

In this embodiment, the conductor lead 114 is attached to the ring by clinching the bulbous end of the tack pin on the lead 114. Initially, one end of the conductor 114 is placed in the slot 120 of the tack pin 113, which has already been soldered to the circuit panel. The clinch ring 117 is then installed on the tack pin with the clinch ring slot 122 aligned with the tack pin slot 120. The tack pin is then pressed into the clinch ring 117 in the same manner as described above. When the tack pin 113 is pressed into the clinch ring 117, the walls of the tack pin 113 adjacent the slot 120 compress on the lead 114 and hold it in place. Additionally, metal from the clinch ring 117 also enters the slot 120 above the wire to hold the conductor even tighter. The combination of the tack pin walls compressing the lead 114 and metal flowing above and contacting the lead 114 creates a solid and reliable mechanical and electrical connection.

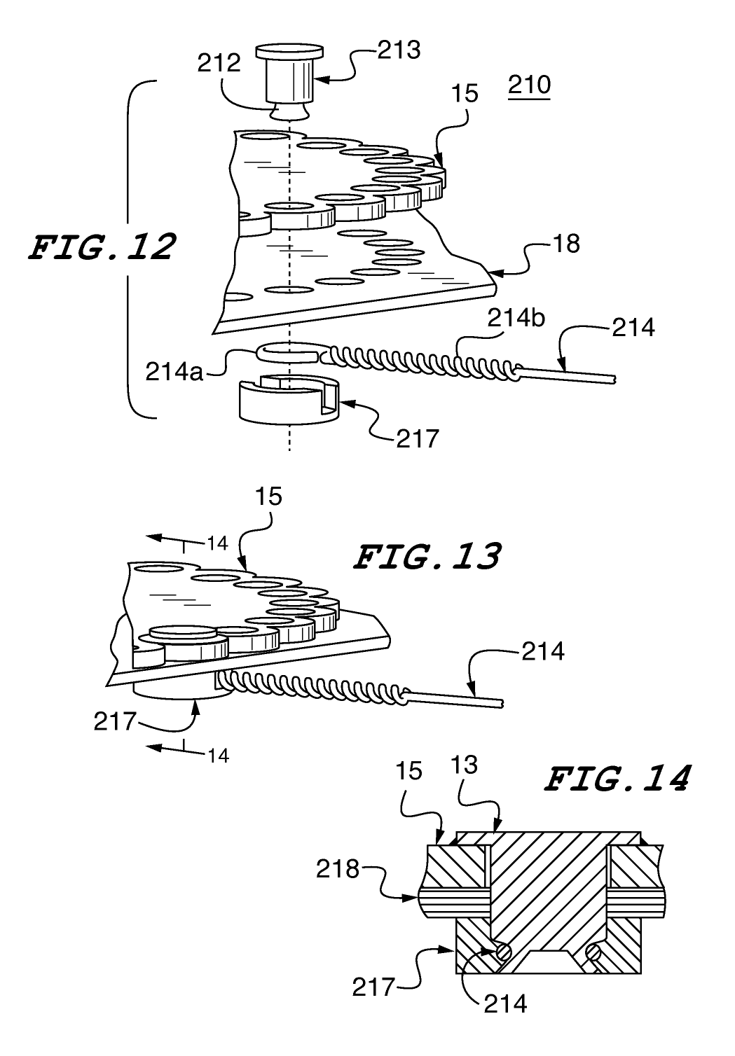

FIGS. 12 through 14 show an additional preferred embodiment of the invention and is designated generally by reference numeral 210. The connector of the assembly mechanically and electrically joins a wearable electronic 15 to a fabric layer 18. The connector 210 generally comprises a tack pin 213, a metal clinch ring 217 and a conductor lead 214. The tack pin 213 has a head 213a, a shank 213b, an outwardly-flared end 213c, and an undercut 212 intermediate the head 213a and the flared end 213c. The tack pin 213 has a construction similar to the tack pin 13 illustrated and described above with respect to FIGS. 1-7. However, in this embodiment, the distal (relative to the head) end of the tack pin 213 has a frusto-conical shape instead of a bulbous shape.

In this preferred embodiment, the clinch ring 217 has the same construction as the clinch ring 217 illustrated and described above with reference to FIGS. 8-11. The clinch ring 217 has a central, cylindrical bore, annular, flat top and bottom surfaces 217b, 217c, respectively, perimeter surface 217d, and a slot 222 in the top surface.

The conduction lead 214 comprises a strand of metal wire having a circular cross section, a looped end 214a and a coiled midsection 214b. As best seen in FIGS. 9 and 11, the diameter of the wire is slightly smaller than the width of the slots 220, 222.

In this embodiment, the conductor lead 214 is attached to the clinch ring 217 by clinching the ring on the lead 214. Initially, one end of the lead 214 is looped around and wrapped in the undercut 212 of the tack pin 213 in the undercut. The clinch ring 217 is then installed on the tack pin with the clinch ring slot 222 aligned with the axis of the lead 214. The tack pin is then pressed into the clinch ring 217 in the same manner as described above. When the tack pin 213 is pressed into the clinch ring 217, the metal from the clinch ring 217 is cold deformed, pushed into the undercut 212 and compressed against the looped end 214a of the lead 214. Metal flowing into the undercut creates a solid and reliable mechanical and electrical connection.

From the foregoing disclosure, one of skill in the art will understand that the invention provides the capability of high speed production of both physical and electrical integration of electronic circuitry into clothing and other types of garments. There may be modifications to the invention that will be available to those of skill in the art without departing from the spirit and scope of the invention which shall be defined by the following claims and their legal equivalents.

1. An electronic assembly for a garment, comprising, from top to bottom:
a tack pin having a head, a shank, and clinch attachment means with an undercut for receiving the cold flow of metal;
an electronic circuit board having a top surface in contact with the head of the tack pin and a hole through which the tack pin downwardly extends;
a layer of fabric beneath the circuit board which the shank of the tack pin passes through;
a metal ring disposed beneath the fabric layer into which said tack pin is clinched by forcing material from the ring into the undercut; and
a wire attached to the ring being electrically connected to the electronic circuit board.

2. The assembly of claim 1 wherein the tack pin is one of a plurality of tack pins which extend through holes in the circuit board to a plurality of rings and wires attached to each of the rings.

3. The assembly of claim 1 wherein the fabric layer is a portion of a garment.

4. The assembly of claim 1 wherein the tack pin shank has a slot at the bottom through which the wire passes.

5. The assembly of claim 1 wherein the ring has a peripheral groove occupied by the wire.

6. The assembly of claim 1 wherein the wire occupies the undercut.

7. The assembly of claim 1 wherein the bottom end of the tack pin is flush with a bottom of the ring.

8. The assembly of claim 1 wherein the tack pin is electrically connected to the circuit board by surface mount soldering.

9. The assembly of claim 8 wherein electricity is conducted between the circuit board and another device which transmits signals through the wire to the circuit board.

10. The assembly of claim 4 wherein the ring has a second slot in alignment with the tack pin shank slot.

11. The assembly of claim 2 wherein the circuit board is circular and a plurality of the tack pins are disposed about a periphery of the circuit board.

12. The assembly of claim 11 wherein a plurality of the wires are soldered each to one of a plurality of the rings and extend radially outward from the circuit board.
